(19) United States
(12) Patent Application Publication
(10) Pub. No.: US 2012/0259224 A1
(43) Pub. Date: Oct. 11, 2012

(54) Ultrasound Machine for Improved Longitudinal Tissue Analysis

(76) Inventors: Mon-Ju Wu, Madison, WI (US); William A. Sethares, Madison, WI (US); Ray Vanderby, Madison, WI (US)

(21) Appl. No.: 13/083,038

(22) Filed: Apr. 8, 2011

(51) Int. Cl. A61B 8/00

(52) U.S. Cl. 600/443

(57) ABSTRACT

An ultrasound machine provides for segmentation of tissue structure that may track isolated tissue structures over multiple frames of data taken over time. Analysis of the isolated tissue structure permits better discrimination of small differences between tissue structures such as may indicate tissue damage or disease.

### STATEMENT REGARDING FEDERALLY SPONSORED RESEARCH OR DEVELOPMENT

This invention was made with government support under EB008548 awarded by the National Institutes of Health. The government has certain rights in the invention.

### CROSS REFERENCE TO RELATED APPLICATION

### Background of the Invention

The present invention relates to ultrasonic imaging equipment and in particular to a method and apparatus for providing improved measurements of subtle changes in tissue as a function of time.

Conventional ultrasonic imaging provides a mapping of ultrasonic echo signals into an image where the intensity of the echo, caused principally by relatively small differences in material properties between adjacent material types, is mapped to brightness of pixels on the image plane. While such images serve to distinguish rough structure within the body, they provide limited insight into the physical properties of the imaged materials.

Newly developed ultrasonic imaging machines may measure physical properties of the imaged materials to reveal stiffness properties of the material. Such imaging is sometimes referred to as “elastography”.

In one type of elastography, termed “quasi-static” elastography, two images of a material in two different states of compression, for example no compression and a given positive compression, may be obtained by the ultrasound device. The material may be compressed by a probe (including the transducer itself) or, for biological materials, by muscular action or movement of adjacent organs. Strain may be deduced from these two images by computing gradients of the relative shift of the material in the two images along the compression axis. Quasi-static elastography is analogous to a physician's palpation of tissue in which the physician determines stiffness by pressing the material and detecting the amount of material yield (strain) under this pressure.

U.S. Pat. Nos. 7,736,315; 7,744,535 and U.S. patent application 2010/0228125, all assigned to the assignee of the present invention, describe “acoustoelastic” techniques to measure mechanical tissue properties which, rather than deducing strain by measuring the motion of the material, deduces strain directly from the modification of the ultrasonic signal caused by changes in the acoustic properties of the material under deformation, for example the change in reflected energy in the ultrasonic signal. In situations when the strain is known, this technique may be used to derive serial properties related to the elasticity of the tissue such as Poisson's ratio, Young's modulus, and other common strain and strain-related measurements.

### SUMMARY OF THE INVENTION

The present invention provides a method and apparatus to automatically segment or isolate tissue being studied by ultrasonic techniques so that the same tissue can be identified at different times. When combined with acoustoelastic techniques, the segmentation permits the detection of subtle changes in acoustic properties that would otherwise be lost if averaged with the acoustic properties of surrounding tissue. The present invention raises the possibility of new diagnostic procedures based on measurement of tissue properties of extremely small tissue regions, for example portions of tendons or the wall of a blood vessel. The possibility of accurately monitoring the elastic properties of a blood vessel wall presents the possibility of early detection of vascular disease.

Specifically the present invention may provide an ultrasound machine including an ultrasonic signal acquisition system adapted to transmit an ultrasonic signal into a body and to receive and measure the ultrasonic signal as modified by tissue of the body, the measurements providing a series of data points in at least two spatial dimensions. An electronic computer communicating with the ultrasound signal acquisition system may execute a stored program to receive measurements from the ultrasonic signal acquisition system to: (a) automatically identify a preliminary spatial division of the data points into internal data points within the tissue structure, external data points outside of the tissue structure, and at least some uncommitted intermediate data points between the internal and external data points; (b) determine at least one dividing criterion from the internal data points and external data points; (c) assign the uncommitted data points to one of the internal data points and external data points using the dividing criterion; and (d) determine a property of the tissue structure based on combined value of internal data points after application of the dividing criterion.

It is thus a feature of at least one embodiment of the invention to provide an automatic segmentation tool that derives a threshold not simply from data within the region of interest but also from data outside. It is another feature of at least one embodiment of the invention to provide a segmentation system that more broadly considers the statistics of the entire region of interest in making local thresholding decisions.

After step (c), step (b) may be repeated to determine a second dividing criterion and step (c) may also be repeated to assign the data points according to the second dividing criterion.

It is thus a feature of at least one embodiment of the invention to provide an iterative method of refining the segmentation.

Step (a) may include the steps of receiving at least one seed region holding multiple data points within the tissue structure and automatically identifying internal data points based the seed region.

It is thus a feature of at least one embodiment of the invention to provide a simple method of identifying a region to be segmented by simply selecting a few points within that region.

The seed region may be expanded in two steps to provide uncommitted data points in a first region surrounding the seed region and external data points in a second region surrounding the uncommitted data points.

It is thus a feature of at least one embodiment of the invention to provide for relatively “pure” internal and external regions that may be used to define a threshold value for the region of interest.

The expansion of the seed region substantially preserves the shape of the seed region.

It is thus a feature of at least one embodiment of the invention to provide an automatic method of identifying an approximate boundary of the region that is neither over- nor under-representative of the internal or external regions.

The initial seed region maybe identified manually on a standard ultrasound image.

It is thus a feature of at least one embodiment of the invention to provide a flexible method of region identification that may work with a variety of different region types.

The data points may provide measures of echo strength of the received ultrasonic signal from tissue at those points and the dividing criterion may be an echo strength criterion. In one example, the data points may provide measures of acoustoelastic properties of the tissue at those points and the dividing criterion is an acoustoelastic criterion.

It is thus a feature of at least one embodiment of the invention to provide a segmentation system that may consider segmentations based on properties of the tissue other than texture or other spatial features.

The ultrasonic apparatus may output data indicating acoustoelastic properties of the internal data points.

It is thus a feature of at least one embodiment of the invention to provide a system that may augment sensitive measurements of acoustoelastic properties.

The ultrasonic acquisition system provides a time series of data frames each holding data points in at least two spatial dimensions and further includes the steps of: projecting a region of interest defined by the internal data points of a first frame at step (c) to a second frame to define internal and external data points in the second frame, and performing steps (b)-(c) for the second data frame using the defined internal and external data points of the second frame.

It is thus a feature of at least one embodiment of the invention to provide an automatic segmentation system that may identify the same regions of interest in the multiple images over time to permit longitudinal studies of the tissue.

The ultrasonic apparatus may further track motion between the data frames and the electronic computer may further execute the stored program to project the region of interest between the first and second data frame according to a determined motion between the first and second data frame. The tracking of motion may be performed by a shifting between the first and second frame providing a best matching of the data associated with first and second frames.

It is thus a feature of at least one embodiment of the invention to employ motion-tracking techniques to improve the segmentation among different data frames taken of moving tissue.

The region of interest of the first frame may be shrunken contemporaneously with projection onto the second frame.

It is thus a feature of at least one embodiment of the invention to permit the auto segmentation process to correct for changes in region dimension and motion.

The ultrasonic apparatus may employ the dividing criterion of the first frame in identifying internal data points in the second frame.

It is thus a feature of at least one embodiment of the invention to accelerate the segmentation process in later frames by using the dividing criteria of the previous frame in the first iteration of the segmentation.

The dividing criterion may describe a hyper plane or some other simply parameterized separating surface.

It is thus a feature of at least one embodiment of the invention to permit sophisticated multiparameter segmentation of a region of interest.

The dividing criterion may evaluate multiple moments of the data points.

It is thus a feature of at least one embodiment of the invention to provide a versatile framework for the dividing criteria.

The ultrasonic acquisition system may provide a time series of data frames each holding data points in at least two spatial dimensions and the electronic computer may further execute the stored program to output a data value indicating changes in property of the tissue structure across at least two data frames.

It is thus a feature of at least one embodiment of the invention to permit tissue to be characterized with respect to a change in properties under different tissue conditions, for example tension.

Generally the present invention provides a method of tissue analysis comprising the steps of transmitting an ultrasonic signal into a body and receiving and measuring the ultrasonic signal as modified by tissue of the body, the measurements to produce a time series of data frames each comprising data points in at least two spatial dimensions at different times. The method may employ an electronic computer to identify a tissue structure of interest within the body in different data frames and determine a change in an acoustic property of the tissue structure between data frames based on combined value of data points within the tissue structure.

It is thus a feature of at least one embodiment of the invention to permit improved sensitivity in the measurement of acoustoelastic properties by accurate segmentation of a region of interest.

In two important embodiments, the tissue structure may be a tendon and blood vessel wall.

It is thus a feature of at least one embodiment of the invention to provide improved measurements of these tissue structures in vivo.

The comparison performed on the blood vessel wall may be done at predetermined phases of the cardiac cycle and the process may include the step of outputting an indication of vascular disease.

It is thus a feature of at least one embodiment of the invention to provide a new and sensitive measure of blood vessel health that may be performed by ultrasound equipment.

These particular features and advantages may apply to only some embodiments falling within the claims and thus do not define the scope of the invention. The following description and figures illustrate a preferred embodiment of the invention. Such an embodiment does not necessarily represent the full scope of the invention, however. Furthermore, some embodiments may include only parts of a preferred embodiment. Therefore, reference must be made to the claims for interpreting the scope of the invention.

### BRIEF DESCRIPTION OF THE FIGURES

FIG. 1 is a block diagram of an ultrasound machine suitable for practice of the present invention;

FIG. 2 is a simplified representation of multiple data frames of ultrasonic data as may be used by the present invention;

FIG. 3a is a simplified view of a tendon showing a region of interest with the tendon in a relaxed state;

FIG. 3b is a figure similar to that of FIG. 3a showing the tendon in a state of tension causing movement of the region of interest such as may be accommodated by the present invention;

FIG. 4a is a chart showing a first segregation of the data points of the data frame of FIG. 2 into internal and external regions and the establishment of the dividing criterion to segment data points that have not been identified to be internal or external regions;

FIG. 4b is a figure similar to that of FIG. 4a showing a refined division between data points fully segregated into the internal and external regions;

FIG. 5 is a flowchart linked to multiple diagrams depicting the operation of the segmentation system of the present invention;

FIG. 6 is a simplified representation of an initial region of interest showing its expansion into multiple regions as used in the process of FIG. 5; and

FIG. 7 is a diagram showing use of the invention in the measurement of vascular health.

### DETAILED DESCRIPTION OF THE PREFERRED EMBODIMENT

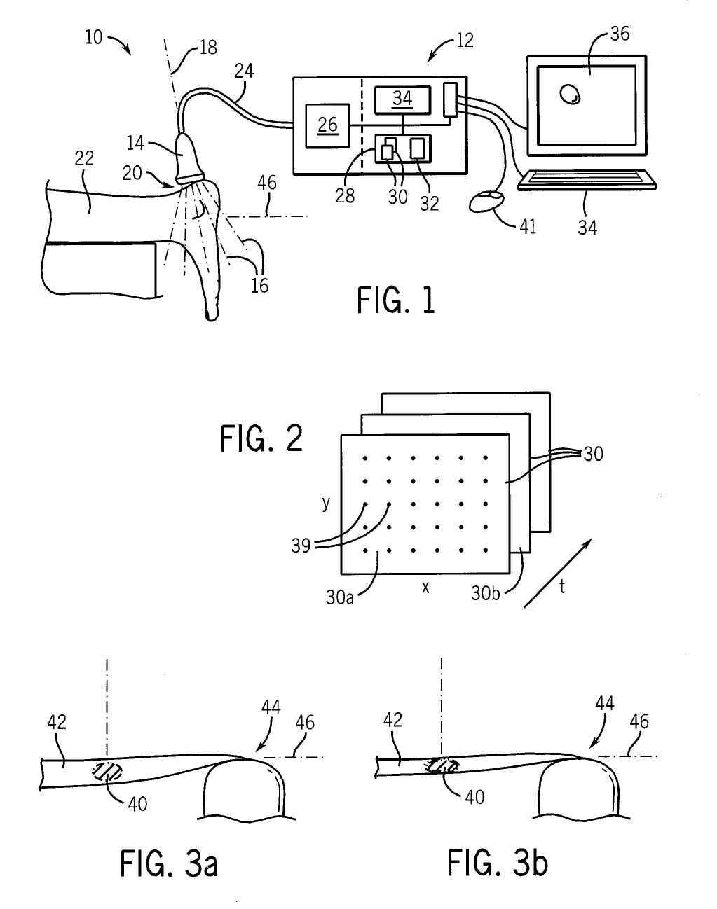

Referring now to FIG. 1, an ultrasound apparatus 10 suitable for use with the present invention may employ an ultrasonic imaging machine 12 providing the necessary hardware and/or software to collect and process ultrasonic echo signals. During operation of the ultrasonic imaging machine 12, an ultrasonic transducer 14 may transmit an ultrasound beam 16 along an axis 18 toward a region of interest 20 within a patient 22 to produce echo signals returning generally along axis 18. The echo signals may be received by the ultrasonic transducer 14 and converted to an electrical echo signal.

The electrical echo signals may be communicated along lead 24 to be received by interface circuitry 26 of the ultrasonic imaging machine 12, the former providing amplification, digitization, and other signal processing of the electrical signal as is understood in the art of ultrasonic imaging.

Referring also to FIG. 2, digitized echo signals may then be transmitted to a memory 28 for storage in multiple data frames 30 representing multidimensional data acquired at separate sequential times. Generally, each data frame 30 will be comprised of multiple data points 39 arranged over two or three dimensions (two dimensions shown for clarity) corresponding to physical locations within the patient tissue. Each data point 39 will thus have a set of coordinate values describing its dimensional location in space and a data value being a measure of the echo signal at that coordinate. The data value may be the strength of the echo or other measures of the echo signal (e.g. phase or spectrum) and alternatively or in addition be further processed to provide, for example, the data values that indicate material properties of the tissue at those coordinates, for example stiffness, strain or the like, using acoustoelastic calculations. In the present example, it will be assumed that each data value is associated with both a B-mode image data value, and an acoustoelastic data value which may be used interchangeably as described. Generally, multiple data frames 30 will be obtained at sequential points in time.

The data frames 30 as stored in memory will be processed according to a stored program 32 of the present invention by a processor 34 as will be described below.

After processing, the data points 39 of the data frames 30 may be used to construct an image displayed on graphical display 36 (for example an image indicating tissue properties) or may be displayed quantitatively on the graphical display 36. The term “image” here is used generally to indicate a mapping of data values to pixel values according to the coordinates of the data values and need not be a conventional ultrasound image.

Input commands affecting the display of the data points 39 and their processing may be received via a keyboard 38 or cursor control device 41, such as a mouse, attached to the processor 33 via interface 26, as is well understood in the art.

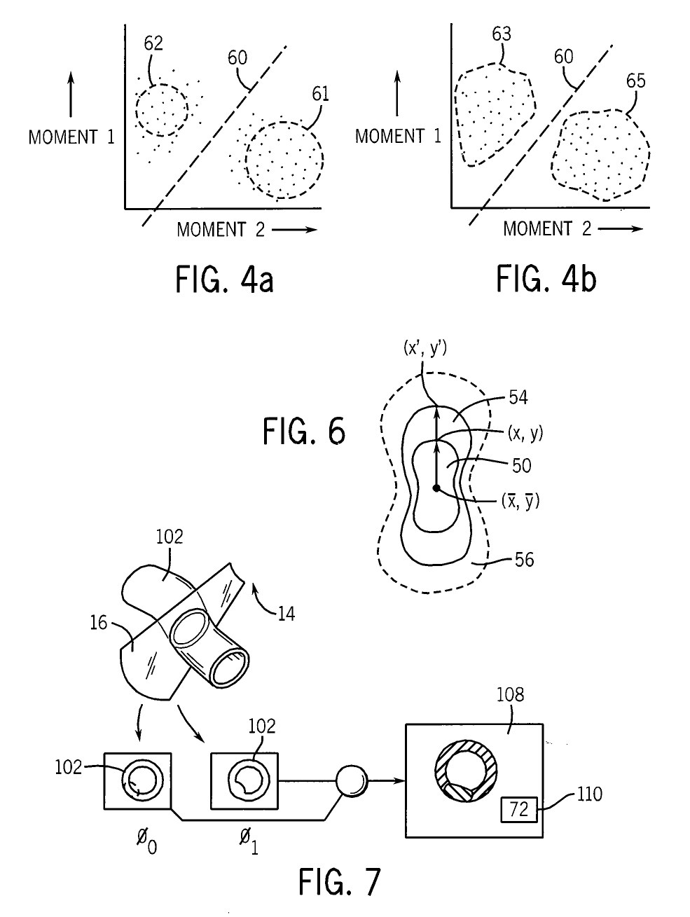

Referring now to FIGS. 1, 4a and 4b, in one nonlimiting application, the invention may be used for analyzing a region of interest 40 in the Achilles' tendon 42 of the human heel 44. The region of interest 40 is selected to represent tissue being qualified or evaluated, for example, for injury or disease. During that analysis, different data frames 30 may be obtained at different times with the tendon 42 in different states of tension, for example, by instructing the patient to press down on the ball of the foot against a restraining force or scale. The tension to the tendon 42 may be applied along axis 46 generally perpendicular and crossing axis 18 of the ultrasonic beam.

In these different tension states, represented by FIGS. 4a and 4b, the region of interest 40 may move and change in size and shape. Precise quantitative evaluation of the region of interest 40 requires isolation or “segmentation” of the region of interest in each of the data frames 30 so that the data points 39 within the region of interest may be processed in isolation of other tissue. This allows properties intrinsic to the region of interest to be distinguished (and separated from) adjacent tissues. For example, only the data values within the region of interest 40 could be averaged or otherwise processed. Referring again to FIG. 1, the program 32 of the ultrasonic imaging machine 12 may provide this segmentation for successive sequential data frames represented, for example, by data frame 30a and data frame 30b.

Referring now to FIG. 1 and FIG. 5, in a first step of the program 32, as indicated by process block 48, a seed internal region 50 is defined in an initial data frame 30. This identification can be performed manually, for example, on a B-mode image of the data frame 30a displayed on display 36 (shown in FIG. 1). In one embodiment, a physician may simply draw the seed internal region 50 by specifying a small number of points within the desired region of interest 40 as displayed, whose internal area may be mapped to the actual data points in the data frame 30 to identify the data points 39 within the seed internal region 50. Desirably the seed internal region 50 is placed comfortably inside the actual region of interest 40.

Referring to FIGS. 5 and 6, at succeeding process block 52, this seed internal region 50 is expanded to define an intermediate region 54 surrounding the seed internal region 50, and an external region 56 surrounding the intermediate region 54. The amount of expansion may be chosen to be a uniform percentage in all directions about the center of the seed internal region 50 to largely preserve the shape of the seed internal region 50.

In one embodiment, an expansion ratio is used to create intermediate region 54 by enlarging the seed internal region 50. Suppose (x, y) is a boundary pixel of the internal region, ( x̅ y̅) is the center of the interior region, and (x′, y′) is the boundary pixel of the enlarged region with the same normalized vector to ( x̅, y̅) as point (x, y). The location of (x′, y′) and the expansion ratio can be determined by the following equations:

$$\begin{matrix}\frac{( {x}^{′} - \overline{x} , {y}^{′} - \overline{y} )}{\sqrt{{( {x}^{′} - \overline{x} )}^{2} + {( {y}^{′} - \overline{y} )}^{2}}} = \frac{( x - \overline{x} , y - \overline{y} )}{\sqrt{{( x - \overline{x} )}^{2} + {( y - \overline{y} )}^{2}}} & ( 1 ) \\ Expansion Ratio = \frac{\sqrt{{( {x}^{′} - \overline{x} )}^{2} + {( {y}^{′} - \overline{y} )}^{2}}}{\sqrt{{( x - \overline{x} )}^{2} + {( y - \overline{y} )}^{2}}} & ( 2 )\end{matrix}$$

Let P_{INT} be the data set of the location of points in the seed internal region 50. Applying an expansion ratio, IR creates a larger region P_{A} (intermediate region 54) that encloses the internal region P_{INT} (seed internal region 50). The intermediate region P_{MID} is given by:

P_{A}=IR×P_{INT} (3)

P_{MID}=[(x,y)∈P_{A}]∩[(x,y)∉P_{INT}] (4)

The second expansion ratio ER is applied to create another region P_{B} (external region 56) that encloses both the internal region and the intermediate region. The external region P_{EXT} is given by:

P_{B}=ER×P_{INT} (5)

P_{EXT}=[(x,y)∈P_{B}]∩[(x,y)∉P_{A}] (6)

In one embodiment, the expansion ratio IR is initialized to 1.3 while the expansion ratio ER of biomedical tissue may lie between 2.5 and 3 (though it may vary with different tissue characteristics). The expansion ratio may be adjusted automatically in order to ultimately fit the data.

Referring again to FIG. 5, at process block 58, the data points 39 in each of the seed internal region 50 and the external region 56 are used to develop a dividing criterion. This process considers both the spatial location of the data points 39 within each of the seed internal region 50 and external region 56 and the values of the data points (either B-mode or acoustoelastic value).

The dividing process in one embodiment considers not only the values of the data points 39 themselves but statistical features of the data points 39. The particular statistical features may be a collection of moments, which are functions of the pixel intensity and different orders of distance. These are adapted from R. C. Gonzales and R. D. Woods, Digital Image Processing, Third Edition, Prentice Hall, 2008.

$${μ}_{PQ} = {\sum }_{y = \overline{y} - 2}^{y = \overline{y} + 2} {\sum }_{x = \overline{x} - 2}^{x = \overline{x} + 2} {( x - \overline{x} )}^{P} {( y - \overline{y} )}^{Q} f ( x , y ) \qquad ( 8 )$$

Suppose x̅ and y̅ is the location of the pixel, x and y is the location of each surrounding pixel (in a 5 by 5 neighborhood in this example), and f(x, y) is the intensity of point (x, y). P and Q are the numbers of order of the distance factor, which usually varies from 0 to 3.

$$\begin{matrix}{μ}_{00} = {\sum }_{y = \overline{y} - 2}^{y = \overline{y} + 2} {\sum }_{x = \overline{x} - 2}^{x = \overline{x} + 2} {( x - \overline{x} )}^{0} {( y - \overline{y} )}^{0} f ( x , y ) & ( 9 ) \\ {μ}_{20} = {\sum }_{y = \overline{y} - 2}^{y = \overline{y} + 2} {\sum }_{x = \overline{x} - 2}^{x = \overline{x} + 2} {( x - \overline{x} )}^{2} {( y - \overline{y} )}^{0} f ( x , y ) & ( 10 ) \\ {μ}_{02} = {\sum }_{y = \overline{y} - 2}^{y = \overline{y} + 2} {\sum }_{x = \overline{x} - 2}^{x = \overline{x} + 2} {( x - \overline{x} )}^{0} {( y - \overline{y} )}^{2} f ( x , y ) & ( 11 )\end{matrix}$$

With these moments, the normalized central moments, denoted as η_{pq}, can be defined as

$${η}_{PQ} = \frac{{μ}_{PQ}}{{μ}_{00}^{r}} \\ where \\ r = \frac{P + Q}{2} + 1 \qquad ( 12 )$$

A set of invariant moments can be derived from the second and third moments:

φ₁=η₂₀+η₀₂ (13)

φ₂=(η₂₀−η₀₂)²+4η₁₁² (14)

φ₃=(η₃₀−3η₁₂)²+(3η₂₁−η₀₃)² (15)

φ₄=(η₃₀+η₁₂)²+(η₂₁+η₀₃)² (16)

The algorithm uses a five by five matrix centering at each pixel and calculates the seven moments (μ₀₀, μ₂₀, μ₀₂, φ₁, φ₂, φ₁φ₃, φ₄) within this matrix. This helps prevent the influence of inconsistent speckles and other irregularities which are often noise.

The moments associated with each data point 39 may be represented graphically as shown in FIG. 4a (only two moments shown for clarity) with the data points 39 of the seed internal region 50 shown in a first cluster 61 and data points 39 of the external region 56 shown in cluster 62. Generally, the statistics of the data points 39 of the intermediate region 54 will be distributed both inside and outside of the clusters 61 and 62.

The moments associated with each data point 39 are then processed to determine a dividing boundary 60 that will be used as a dividing criterion between data points 39 within the region of interest 40 and outside of the region of interest 40.

One method of making the dividing boundary 60 compares the differences in the statistical features of the two regions: seed internal region 50 and external region 56, and calculates an empirical value through minimum squared-error and pseudoinverse equation, as used for pattern classification in R. O. Duda, P. E. Hart, and D. G. Stork, Pattern Classification, 2nd Edition, John Wiley and Sons, 2001. Generally, for multiple moments, the dividing boundary 60 may be a hyperplane, however it is also possible to use high-dimensional surfaces (i.e. greater than three dimensions) other than a hyperplane, for example, quadratic surfaces as also taught in the above reference, or Gaussian surfaces

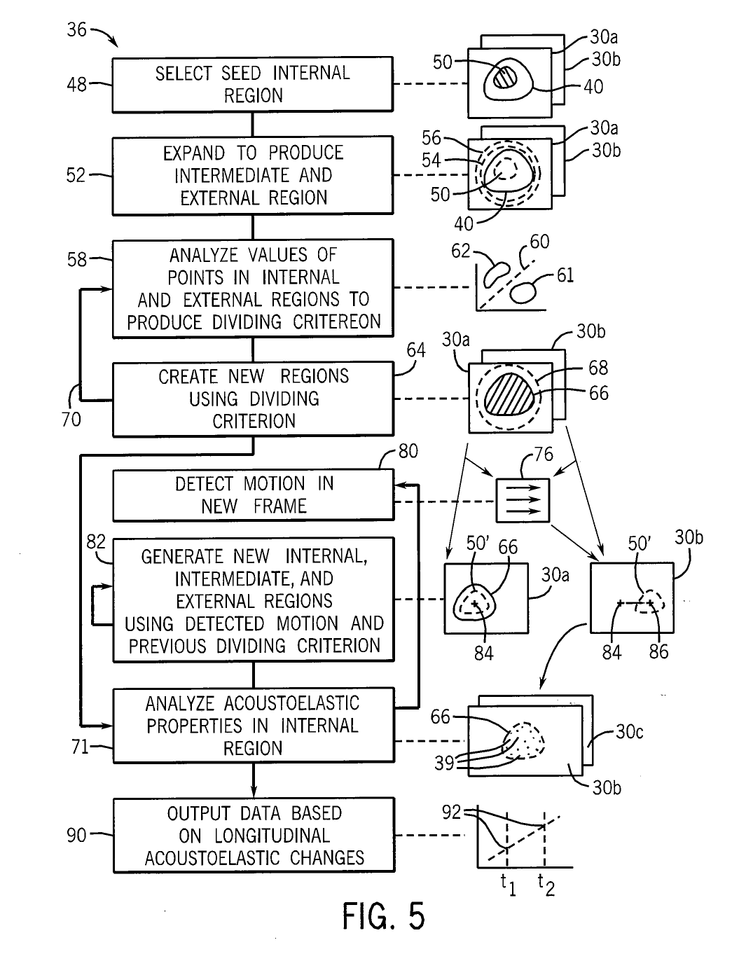

Referring again to FIGS. 3b and 5, the dividing boundary 60 of process block 58 is used as indicated by process block 64 to divide the remaining data points 39 into new clusters 63 associated with the region of interest 40 and cluster 65 associated with points outside of the region of interest 40. This new division of the data points 39 provides a refined seed region 66 and refined external region 68 the latter having a shared outer boundary with originally defined external region 56.

A loop 70 is provided so that the analysis of process block 58 is repeated using this new dividing boundary 60 and new refined seed region 66 and refined external region 68 of process block 64 to recompute the dividing boundary 60. This refinement of the dividing boundary 60 may be repeated for multiple iterations.

The data points 39 identified to the ultimate refined seed region 66 closely approximating the region of interest 40 may then be used as indicated by process block 75 for analysis of acoustoelastic properties of the region of interest 40. By isolating the region of interest 40 from other tissue, sensitive measurements of the region of interest may be extracted. This extraction process may for example combine the values of the data points 39 in the region of interest 40 to reduce the effects of noise and the like.

Referring still to FIG. 5, as noted above, multiple data frames 30a and 30b may be acquired at sequential times (for example being representative of a set of multiple data frames 30). In order to speed the processing of multiple data frames, a “projection” process is used in which information derived from an earlier data frame (e.g. data frame 30a) is used to inform the processing of the subsequent data frame (e.g. data frame 30b).

For the purpose of making the projection, the underlying image data of the data frames 30a and 30b maybe compared, for example, by correlation, as indicated by process block 80, to produce a set of motion vectors 76 indicating relative motion of the tissue elements between data frame 30a and data frame 30b. It will be understood that the correlation process may use both strict mathematical correlation and other similar correlation type techniques, for example those using sums of different magnitudes. The data being correlated may be standard B-mode data or acoustoelastic data.

The motion vectors 76 are used to project refined seed region 66 having center 84 in data frame 30a to data frames 30b where it becomes seed internal region 50′. The seed internal region 50′ has a center 86 in data frame 30b displaced from center 84 in data frame 30a according to the motion vector 76. In addition, the seed internal region 50′ is a contracted form of region 66 using a contraction process analogous to the expansion process described above.

The seed internal region 50′ is then expanded as described above with respect to process block 52, and the dividing criterion developed for data frame 30a is used to sort data points 39 in the intermediate region 54 per process block 58. This sorting is then used to create a new dividing criterion analogous to process block 64 to finalize the seed internal region 50′ as a refined seed region 66′. Again, at process block 71 the data points 39 within the refined seed region 66′ may be analyzed to accurately characterize acoustoelastic properties.

The process of process block 80, 82 and 71 maybe then repeated for succeeding data frame 30c as desired.

At process block 90, acoustoelastic properties 92 derived from process block 71 for different data frames 30 under different tissue conditions may be analyzed to extract additional information using the techniques described in the previously cited patent references. The tendon 42 (shown in FIG. 4), in this example, could be intentionally stressed by muscle contraction by the patient between two times (for example by pressure on the foot of a known force) to vary tension on the tendon 42 to assess the elasticity of the tissue and its health.

Referring now to FIG. 6, the defined region of interest may be, for example, a cross-sectional slice through an artery 102 at a first phase φ0 of the cardiac cycle exhibiting a first pressure and the second phase φ1 of the cardiac cycle exhibiting a second pressure, the pressure revealing a circumferential tension on the wall of the artery 102. These two measurements may be used together, further references described above, to deduce the elastic properties of the artery 102 such as may reveal early indications of arteriosclerosis. This information may be provided through an image 108, for example, having shading indicating elastic properties, as well as a quantitative regime output 110.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof; and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a controller” and “a processor” can be understood to include one or more controllers or processors that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

We claim:

1. An ultrasonic apparatus comprising:
an ultrasonic signal acquisition system adapted to transmit an ultrasonic signal into a body and to receive and measure the ultrasonic signal as modified by tissue of the body, the measurements providing a series of data points in at least two spatial dimensions;
an electronic computer executing a stored program to receive measurements from the ultrasonic signal acquisition system to:
(a) automatically identify a preliminary spatial division of the data points into internal data points within the tissue structure, external data points outside of the tissue structure, and at least some uncommitted intermediate data points between the internal and external data points;
(b) determine at least one dividing criterion from the internal data points and external data points;
(c) assign the uncommitted intermediate data points to one of the internal data points and external data points using the dividing criterion; and
(d) determine a property of the tissue structure based on the combined value of internal data points after application of the dividing criterion.

2. The ultrasonic apparatus of claim 1 wherein the electronic computer further executes the stored program to repeat step (b) after step (c) to determine a second dividing criterion and repeating step (c) to assign the data points according to the second dividing criterion.

3. The ultrasonic apparatus of claim 1 wherein step (a) includes the steps of receiving at least one seed region holding multiple data points within the tissue structure and automatically identifying internal data points based the seed region.

4. The ultrasonic apparatus of claim 3 wherein the seed region is expanded in two steps to provide uncommitted data points in a first region surrounding the seed region and external data points in a second region surrounding the uncommitted data points.

5. The ultrasonic apparatus of claim 4 wherein the expansion of the seed region substantially preserves the shape of the seed region.

6. The ultrasonic apparatus of claim 3 wherein the seed region is identified manually on a standard ultrasound image.

7. The ultrasonic apparatus of claim 1 wherein the data points provide measures of echo strength of a received ultrasonic signal from tissue at those points and the dividing criterion is an echo strength criterion.

8. The ultrasonic apparatus of claim 1 wherein the data points provide measures of acoustoelastic properties of the tissue at those points and the dividing criterion is an acoustoelastic criterion.

9. The ultrasonic apparatus of claim 1 wherein the electronic computer further executes the stored program to output data indicating acoustoelastic properties of the internal data points.

10. The ultrasonic apparatus of claim 1 wherein the ultrasonic acquisition system provides a time series of data frames each holding data points in at least two spatial dimensions and wherein the electronic computer further executes the stored program to provide the steps of:
projecting a region of interest defined by the internal data points of a first frame at step (c) to a second frame to define internal and external data points in the second frame;
performing steps (b)-(c) for the second data frame using the defined internal and external data points of the second frame.

11. The ultrasonic apparatus of claim 10 wherein the electronic computer further executes the stored program to track motion between the data frames and wherein the step of projecting the region of interest between the first and second data frame projects the region of interest according to a determined motion between the first and second data frame.

12. The ultrasonic apparatus of claim 11 wherein the tracking of motion is performed by determining a shifting between the first and second frame providing a best matching of the data associated with the first and second frames.

13. The ultrasonic apparatus of claim 10 wherein the region of interest of the first frame is shrunken contemporaneously with projection onto the second frame.

14. The ultrasonic apparatus of claim 1 wherein the ultrasonic acquisition system provides a time series of data frames each holding data points in at least two spatial dimensions and further including the step of:
employing in the dividing criterion of a first frame in identifying internal data points in a second frame at a later time.

15. The ultrasonic apparatus of claim 1 wherein the dividing criterion describes a surface having more than three dimensions.

16. The ultrasonic apparatus of claim 1 wherein the dividing criterion evaluates multiple moments of the data points.

17. The ultrasonic apparatus of claim 1 wherein the ultrasonic acquisition system provides a time series of data frames each holding data points in at least two spatial dimensions and further including the step of outputting a data value indicating changes in property of the tissue structure across at least two data frames.

18. A method of tissue analysis comprising the steps of:
(a) transmitting an ultrasonic signal into a body;
(b) receiving and measuring the ultrasonic signal as modified by tissue of the body, the measurements to produce a time series of data frames each comprising data points in at least two spatial dimensions at different times;
(c) using an electronic computer to:
(i) identify a tissue structure of interest within the body in different data frames;
(ii) determine a change in an acoustic property of the tissue structure between data frames based on combined value of data points within the tissue structure.

19. The method of tissue analysis of claim 18 wherein the acoustic property is manifested as a change in a strength of reflected acoustic energy.

20. The method of claim 18 wherein the tissue structure is a tendon.

21. The method of claim 18 wherein the tissue structure is a blood vessel wall.

22. The method of claim 21 wherein the comparison is performed between measurements made at predetermined phases of a cardiac cycle.

23. The method of claim 22 further including the step of outputting an indication of vascular disease.
